(19) United States
(12) Patent Application Publication
(10) Pub. No.: US 2011/0172553 A1
(43) Pub. Date: Jul. 14, 2011

(54) QEEG Statistical Low Resolution Tomographic Analysis

(75) Inventors: Erwin R. John, Mamaroneck, NY (US); Leslie S. Prichep, Mamaroneck, NY (US); Robert Isenhart, Brooklyn, NY (US); Leslie S. Prichep, Mamaroneck, NY (US)

(73) Assignee: New York University

(21) Appl. No.: 12/746,103

(22) PCT Filed: Dec. 12, 2008
(86) PCT No.: PCT/US08/86629

§ 371 Date: Jan. 14, 2011

Related U.S. Application Data
(60) Provisional application No. 61/014,640, filed on Dec. 18, 2007.

(51) Int. Cl. A61B 5/0476

(52) U.S. Cl. 600/544

(57) ABSTRACT

A system and method for analyzing electrophysical signals produced by a brain involves comparing a first selected one of the numerical values to control data including one of a self-norm and a population-norm associated with electrophysical activity in a brain region corresponding to a brain region of origin of the electrophysical activity on which the numerical value is based; calculating a standard score for the brain region of origin based on the comparing; and repeating the comparing and calculating operations for a second selected one of the numerical values, the regions of the brain including subcortical regions extending to a brain stem of the brain.

### PRIORITY CLAIM

This application claims priority to U.S. Provisional Application Ser. No. 61/014,640 entitled “QEEG Statistical Low Resolution Tomographic Analysis” filed Dec. 18, 2007. The specification of the above-identified application is incorporated herewith by reference.

### FIELD OF THE INVENTION

The present invention relates to systems and methods for localizing sources within the brain of EEG potentials recorded from the skin (e.g., the scalp).

### BACKGROUND INFORMATION

Prior systems employed for localizing selected brain regions from which EEG potentials detected on the surface of the skin originate include variable resolution electromagnetic tomographic analysis (VARETA-Bosch-Bayard et al 2001) and low resolution electrical tomographic analysis (LORETA-Pascual-Marqui et al 1999). Both of these QEEG brain imaging methods compute, from the scalp recorded EEG voltages from a set of electrodes placed in positions standardized by the so-called 10/20 Electrode Placement System (Jasper 1958), the current density of sources within the brain that are the most plausible generators of the surface-detected voltages. The sources computed by this solution of the inverse problem are represented in a three-dimensional “proportional” space.

The 10/20 System is a “proportional” system, in that it defines what has long been accepted as the internationally standardized method for placement of sensors of brain electrical activity upon the head of a person to overlie predictable regions of the cortical surface of the brain. It is proportional, because the position of each electrode is defined by a position which lies 10% or 20% of the arc distance along measurements of the size of the head from front to back and from side to side. The position is thus not defined in absolute terms such as centimeters, but in relative terms such as percentage of an arc upon an ellipsoidal representation of the top of the head.

Key to the QEEG brain imaging methods is that the computed sources can be located within either a proportional or a centimetric brain space. The source locations thus computed are superimposed upon transaxial, coronal or sagittal slices from a proportional probabilistic MRI atlas. The proportional brain space is sometimes referred to as a “Talairach space”, because a commonly used 3-D neuroanatomical brain atlas has been published (Talairach/Tournaux, Stereotaxic Atlas of the Human Brain, Thieme, Stuttgart, 1988). Sources are depicted as voxels on such slice images with each voxel color-coded to represent the strength of the source. That is, the superimposed location of each computed source voxel upon the MRI slice indicates the location of the source region of the detected EEG potential while the voxel's color indicates a strength of the activity within that voxel (e.g., the current density). Since the position of these voxels can be identified in three-space as X, Y, and Z coordinates and located in a Talairach space, the neuroanatomical identification of the location of a voxel can be ascertained from available proportional neurosurgical atlases.

Prior QEEG brain imaging systems have provide 3-D models of the absolute or relative (% of total) power of the electrical activity within the brain. Interpretation of such images for experimental or clinical diagnostic functions were carried out by medical practitioners or neuroscientists inspecting such 3-D models much as they read fMRI or PET etc data and reaching conclusions based upon their personal skills, experience and knowledge. The source locations of previous versions of LORETA were restricted spatially to the cerebral cortex, and excluded source locations of brain regions lying below the cerebral cortex. Both VARETA and LORETA, as well as published other 3D source localization methods were based upon an electrode array of 19, 21, 32, 64, 128, or 256 electrodes distributed with approximately equal spacing across the upper hemisphere of the ellipsoidal representation of the head model.

### SUMMARY OF THE INVENTION

The present invention is directed to several improvements in LORETA in particular, but can be extended to any of the other source localization methods by a person skilled in the art.

The major innovative features are:

1) Extension of the computable source locations to the brain stem, spanning all the regions within the neurosurgical stereotaxic brain atlas. Since each location is represented by a voxel, this extension results in an increase in the number of voxels from the previous roughly 3500 to about 6900, and in an improvement in voxel resolution from approximately 7 mm cubes to about 5 mm cubes.

2) Transforming all voltage values on the surface to standard or Z-scores, computing the normative distributions of sources for every frequency in the very narrow band frequency spectrum of the EEG (0 to 50 Hz, or more) with appropriate transforms for Gaussianity and age regression, to enable computation of “neurometric LORETA” images with voxel Z-scores and color encoding of the standard score relative to normative distributions for each voxel within the brain at each frequency. This statistical processing of the data will be termed Neurometric Analysis.

3) Modification to enable source localization computation of a “mini-LORETA” from a limited subset of electrodes such as, for example, in a preferred exemplary implementation, an array of several electrodes upon the skin of the forehead at the 10/20 positions known as F7, F8, F1, F2, Fz. Additional electrodes might be placed upon other arbitrarily selected positions or at positions of the 10/20 system or its systematic expansions such as the 10-10 System, etc. but it is important to define the position of the electrode on the skull/scalp in the 3 dimensional stereotaxic space as measured, for example, by an instrument such as the Polyphemus locator.

4) The system also computes the transfer entropy of the bi-directional informational transactions of electrical current at each of a plurality of frequency ranges between each of a plurality of regions of interest (ROI). That is, the system may compute any or all of the influences of each ROI transmitted to every other ROI, the influences received at each ROI from all other ROIs, and the mutual information received in common by any pair of ROIs from some common third source(s). The LORETA or mini-LORETA transforms this data to Z-scores or standard scores relative to control normative data.

### BRIEF DESCRIPTION OF THE DRAWINGS

FIG. 1 shows an exemplary embodiment of a system for monitoring neurological and cerebrovascular state of a patient.

FIG. 2 shows a two-dimensional image slice of a brain divided into voxels or regions of interest (ROIs) to which respective standard scores are assigned, the standard scores respectively indicating varying degrees of abnormal brain activity for the associated ROIs and being visually represented by colors that represent the varying degrees of abnormal brain activity. A table of numerical values corresponding to the results of the inverse solution can be obtained for all ROIs.

FIG. 3 shows a flow diagram illustrating the operation of the system of FIG. 1.

### DETAILED DESCRIPTION

The present invention may be further understood with reference to the following description and the appended drawings, wherein like elements are provided with the same reference numerals. As described in more detail below, the present invention gathers data from EEG scalp electrodes and determines the strength of contribution of the detected brain activity attributable to each of a plurality of voxels defined within a volume of the brain. Thereafter, the activity in each voxel is subject to neurometric analysis to determine any deviation from expected values and this deviation is expressed as a standard or Z-score available in output from the system. While output can be provided of the numerical Z-score of any voxel at the position in the 3D brain model defined by the coordinates X, Y, Z, ideally the results are depicted as slices from a brain atlas with the Z-scores encoded by a color palette using hues proportional to the probability of abnormality, that is, the significance of the observed deviation from the expected age-appropriate normative value for that frequency in that voxel. The anatomical name of the region in the brain within which any selected voxel is located can be interrogated by placing a cursor upon the point of interest in the display, and is obtained from an anatomical lookup table stored within the QSL (Qstat LORETA) instrument.

The system also computes the transfer entropy of the bi-directional informational transactions of electrical current at each of a plurality of frequency ranges between each of a plurality of regions of interest (ROI), that is, the influences of each ROI transmitted to every other ROI, the influences received at each ROI from all other ROIs, and the mutual information received in common by any pair of ROIs from some common third source(s). The LORETA or mini-LORETA transforms these data to Z-scores or standard scores relative to control normative data. It should be noted that the set of ROIs subsumes a set including all cortical ROIs and, unlike prior art LORETA implementations, a second set of subcortical ROIs. The anatomical elements comprising these ROIs depend upon the configuration of the electrode array in use for the computation. The preferred display of these 3-dimensional transactions may not be a pseudo-brain slice but rather a color coded matrix appropriately encoding for each ROI all of its transmitted influences, all of its received influences and all mutual informational transactions. As would be understood by those skilled in the art, although the application is described with regard to a standard digital EEG device usually incorporating a microprocessor or controlled by a desktop or laptop computer, the invention may also be employed in conjunction with a special purpose portable or handheld EEG system. Those skilled in the art will further understand that, when using the mini-LORETA or any other subset of electrodes, the predictability of results corresponding to brain activity in brain regions adjacent to the electrodes will be reduced under the influence of brain activity in more remote regions of the brain. Healthy levels of activity in these more remote regions will define an upper limit on the predictability of these results (e.g., 75%). Thus, the system may determine, when the predictability of these results exceeds this upper limit that the activity in the more remote regions of the brain has been significantly negatively impacted by, for example, a stroke or other pathology.

Neurometric Analysis is the objective statistical evaluation of the numerical values extracted from electrophysiological signals produced by the central nervous system relative to a control or normative database. The reference database may contain measures of the mean values and standard deviations of a set of QEEG univariate or multivariate variables derived from the population of healthy, normally functioning individuals with entries for various stages of life, preferably extending across the whole human life span or those portions of the life span relevant to a particular inquiry.

Alternatively, the reference database may contain measures of the mean and standard deviation of a set of univariate or multivariate QEEG variables collected from a sufficiently large number of EEG segments from an individual person to be replicable or statistically reliable, to serve as a neurometric “self-norm”. The self-norm can be used to construct LORETA or min-LORETA images of the effects on various brain regions or ROIs in an individual with some condition or in some state of a treatment or procedure intended to correct the condition or alter the state, for example, to examine the changes in pathophysiological brain regions accomplished by a given administered dosage of a specific pharmaceutical agent, or to examine the functional changes in brain regions during the performance of some cognitive or motor task.

Electrophysiological signals produced by the central nervous system exhibit systematic changes as one develops and ages which may be extrapolated from a pool of normative data. Based on these identified systematic changes, patients exhibiting electrophysiological signals that significantly deviate from the systematic behavior have consistently been found to suffer from neurological or psychiatric illness, developmental disorders, cerebrovascular disease, dementia, head injuries, etc. Significant deviations from the normative data rarely occur in normal individuals. Distinctive changes in regional electrophysiological activity may be distinctive for certain kinds of developmental, neurological or psychiatric disorder.

The normative database may, for example, be a set of electrophysiological records compiled from a large number of individuals, of various ages, who exhibit normal development and aging. The normative data may be used to identify the systematic changes within the healthy and normally functioning population. The maturational rate of different neuroanatomical regions can be ascertained for any EEG frequency. The systematic regional developmental maturation of the normative brain images may be quantified to produce analyzing modules and corresponding correlation coefficients for patient diagnosis and/or analysis. Such maturational trajectories may be of clinical utility in evaluation of developmental disorders in children or dementing illness of the elderly.

The standard score or Z-score of the deviation from normative values of voxels in any ROI or different neuroanatomical regions can be ascertained for any EEG frequency, for a patient of any age. The distinctive profile of regional or ROI deviations from the normative voxel values may be quantified and formalized into multivariate discriminant functions to produce analyzing modules that evaluate the probability that the patient is suffering from any one of a large variety of brain dysfunctions characteristic of certain developmental, neurological or psychiatric disorders. Such discriminant classification may be of clinical utility in evaluation of developmental disorders in children, dementing illness of the elderly, and patients in a variety of neurological or psychiatric diagnostic categories.

In the field of EEG “neurometrics”, quantitative electrophysiological measurements (QEEG) are evaluated relative to normative data. Generally, detected analog brain waves, at the microvolt level, are amplified, artifacts removed and the amplified brain waves are converted to digital data. That data is then analyzed in a computer system to extract numerical descriptors which are compared to a set of norms (reference values), either the patient's own prior data (initial state) or a group of normal subjects of the same age (population norm). Such analyses quantify the level of deviation, if any, of the activity of any brain region from the reference values.

A neurometric clinical quantitative EEG (QEEG) acquisition and analysis system may be the Neurometric Analysis System (NAS) which is a proprietary system marketed by NxLink, Inc. The NAS is a system for QEEG analysis which has been made compatible with the formats of digital EEG apparatus produced by almost every major manufacturer of such equipment.

The system according to the present invention acquires and quantitatively analyzes data from, for example, an electroencephalogram (EEG) coupled to a plurality of sensors (e.g., removable EEG electrodes) attached to the scalp, preferably, 19-21 electrodes placed according to the Internationally Standardized 10/20 Placement System. Alternatively as described below, the system may be operated with less electrodes or with electrodes at different placements. The gathered data may then be subjected to automatic artifact removal and other signal processing techniques to identify and/or monitor predetermined features of the EEG.

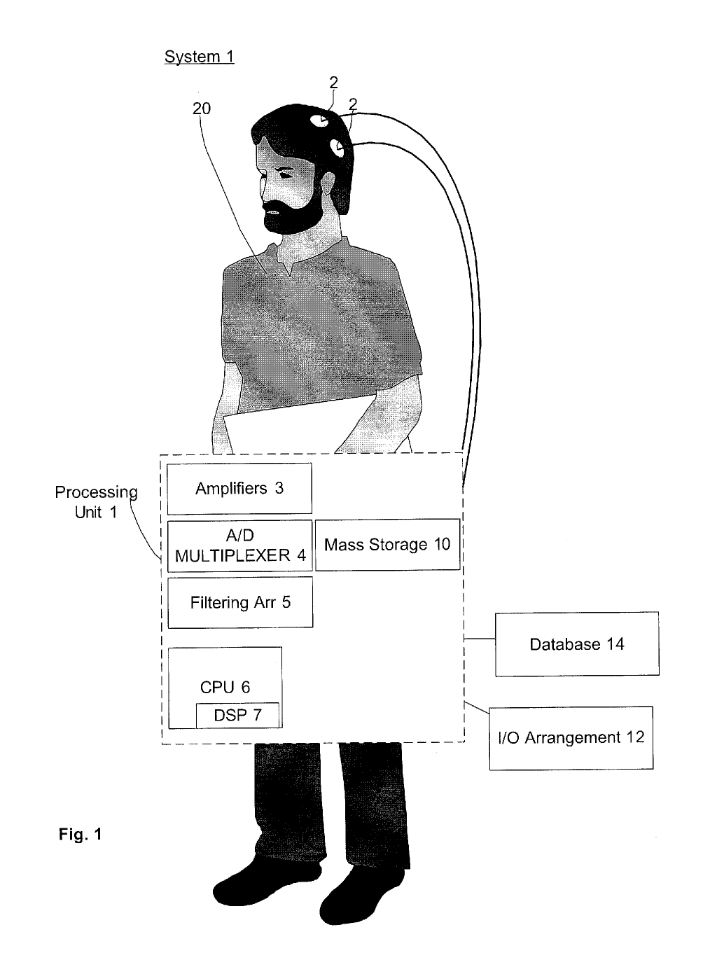

As shown in FIG. 1, a plurality of EEG electrodes 2 (e.g. 19-21 electrodes) are removably secured to a scalp of the patient located in accord with the International 10/20 Electrode Placement System as would be understood by those of skill in the art. Additional removable electrodes may be utilized as desired while additional reference electrodes (unilateral or linked) may be removably positioned on the mastoids or earlobes (A1, A2). Electro-oculogram (EOG) electrodes may optionally be placed at an outer canthus of the eye to facilitate artifact rejection. As would further be understood by those of skill in the art, electrodes may also be placed on the central vertex (Cz) to record brainstem potentials and on the cheekbone to serve as a ground.

The electrodes 2 preferably use a standard electrolyte gel, or other application method, so that the impedance of each electrode-skin contact is below 5000 ohms. Alternatively, for some applications, needle electrodes, a pre-gelled electrode appliance with adhesive or other means of fixation, or an electrode cap or net with previously located electrode positions may be used. The EEG system, described below, automatically checks the electrode-skin impedance at each electrode at frequent intervals, (e.g., every minute), and displays a warning (e.g., a red LED light) if any such impedance falls below 5000 ohms.

Electrode leads connect each of the electrodes 2 to a respective EEG/EP amplifier 3 of a processing unit 1. Each amplifier 3 may preferably include an input isolation switch, (e.g., a photo-diode and LED coupler), to prevent current leakage to the patient. The EEG amplifiers 3 are high-gain low-noise amplifiers, preferably having, for example, peak-to-peak noise of 1 microvolt or less, a band width of 0.1 to 250 Hz, fixed gain of 10,000, common mode rejection of 100 db or more (4 amplifiers). The amplifiers 3 are connected to an analog-to-digital multiplexer 4 (A/D multiplexer). Alternatively, the system may use 24 bit digital amplifiers operating at high sampling rates, for example at 8 to 50 KHz) obviating the need for the A/D, etc. The multiplexer 4 samples the amplified analog brain waves at a rate of, for example, 5-10 KHz for each channel. The multiplexer 4 is connected to a filtering arrangement 5 which is connected to a central processing unit 6 including a dedicated digital signal processor (DSP) 7, such as, for example, model TMS320C44® (Texas Instruments).

A CPU 6 is connected or otherwise has access to a mass storage 10 (e.g., a hard disk), an input/output arrangement 12 including, for example, a keyboard or touch pad and a display such as a CRT or LCD. The software, which comprises a controlled DSP 7, conditions the input signals, insures that the input signals are valid biological signals and performs quality control. Such validity checks on the input signals include periodic calibration measurements and impedance measurements, continuous automatic artifact rejection algorithms, and some means to ensure test-retest replicability, i.e., evidence that the data have converged to a reliable estimate of the values of the set of QEEG variables. The software provides patient information stored, for example, in a patient header including data such as, patient ID number, age and date, a name of a physician, etc. The time is provided by a time code generator, which records both local time and an elapsed time directly on the EEG tracings, so that events may be retrieved from any acquisition session by searching a date in the database. Any data thus retrieved further comprises all clinical protocols and physiological documentation, including the trajectories of the indices. After analysis of the data, the CPU 6 provides information to a display of the I/O arrangement 12 in a format which will be described in greater detail below. Based on the software of DSP 7, the CPU 6 determines QEEG data from the EEG of patient 20 in a manner known to those skilled in the art. Alternatively, rather than calculate QEEG data from a real-time EEG, the QEEG data may be determined from a previously recorded EEG. Under yet another alternative, the QEEG data for patient 20 may have been determined previously, so that CPU 6 performs the statistical analysis of the present invention on the previously recorded QEEG data. Once the QEEG data for patient 20 has been obtained, CPU 6 compares it to the normative QEEG data in database 14. The normative QEEG data may be based on a population norm or a self-norm.

The “relevant” norm is obtained a) from a look-up normative table spanning population norms for persons aged 6 to 90, or b) computed from stored age-regression equations for every QEEG variable, or c) computed from QEEG data previously collected from the subject)) and Z-scores are calculated for the power at each voxel relative to either or both the self- and population-norms. For each voxel, a sliding window, for example, 20 seconds of data which is continuously updated, is formed which integrates sequential segments (i.e., concatenated 1-second artifact-free EEG samples). From the updated mean value of the sliding window, the trajectory of power at each voxel is calculated. The ROIs trajectory of interest for a given application may be user selected or defined, or may be an updated three-dimensional representation relative to the self and population norms, and can be presented to the physician as a quantitative monitor of power at each location within the brain. In the case where a self-norm is used, the self-norm represents QEEG data taken from the patient 20 when the patient was not exhibiting any symptoms indicative of abnormal brain activity or in some reference prior state.

As would be understood by those of skill in the art, the system of FIG. 1 may be implemented incorporating a dedicated freestanding computer, such as a PC, a laptop, a PDA or other handheld device. Furthermore, the system may be implemented in conjunction with a wired or wireless network such as a local area network or the Internet with any of the processing and/or memory storage components located in any of the devices of the system or distributed over a plurality of devices separated from one another. This application permits remote but real-time bedside brain imaging, which may be of clinical value in some conditions.

Alternatively, the computer and digital amplifier portions may be implemented as a module within a multi-modal monitor, which may also include sensors and displays of the patient's vital signs (i.e., blood pressure, respiration, O₂ saturation, temperature and pulse (heart rate). This application permits real-time bedside brain imaging, which may be of clinical value in some conditions.

Alternatively, min-LORETA or selected ROI combinations from several different individuals may be displayed as separate or sequential fields on a monitor which may be, for example, in a multi-bed intensive care unit (ICU) or on a remote monitor conveniently located at some distance for an ICU physician or other ICU personnel. This application permits real-time bedside or operating-table brain imaging, which may be of clinical value in some conditions. This would be desirable if the LORETA or mini-LORETA system of the present invention is used to construct 3D brain images in real time as a way of visualizing the state of brain tissue during radiosurgery, transluminal neuroembolization, or other invasive or non-invasive therapeutic interventions in the brain. In any event, preferably, the display of the I/O arrangement 12 is a monitor having a color screen displaying graphics and alphanumerics while a keyboard, touch screen or other input arrangement preferably includes a standard ASCI key board which may be used to enter the patient header (e.g., name, age, gender, hospital number, date, etc.) and comments (which may use function keys). Preferably, the display shows the results of the LORETA analysis continually updating during the intervention.

In a further embodiment of the invention, the system includes a module analyzing the EEG data to identify and quantify amounts of electrical current (and, therefore, data transfer) between various regions of interest in the brain. For example, using a normative database including data corresponding to a transfer entropy associated with each of a plurality of very narrow band (VNB) frequencies covering a wide demographic spectrum (e.g., subjects ranging from 6 to 90 years of age) allow a comparison of an amount of current being transferred between each of a plurality of contiguous regions of interest (ROIs). That is, the transfer entropy for an ROI corresponds to an amount of current sent to and received from any of the plurality of contiguous ROIs. As would be understood by those skilled in the art, the ROIs are generally in cortical regions subjacent to the various scalp electrodes. For example, in conventional EEG notation: Fp1, Fpz, Fp2, F7, F3, Fz, F4, F8, T3, C3, Cz, C4, T4, T5, P3, Pz, P4, T6, O1, Oz, O2; plus contiguous groups of voxels centered at seven subcortical regions including the Globus Pallidus, Putamen, Caudate, Amygdala, Parahippocampus, Thalamus and Hippocampus. The transfer entropy computational software enables the QSL to calculate from the EEG data recorded from a subject, the bi-directional information transactions at each frequency between all pairs of loci among this set of ROIs. The transfer entropy may be computer using well known mathematical techniques such as, for example, an auto-regressive moving average (ARMA). The computed transfer entropy values for each frequency are then transformed to Z-scores or standard scores relative to the normative transfer entropy values for comparable individuals available from the database.

If a number of electrodes less than the standard International 10/10 System are employed, the device may make use of the covariance matrix at every frequency stored in the memory to compute QEEG source localization images albeit with reduced spatial resolution. For example, as would be understood by those skilled in the art, such localization images may be computed employing Singular Value Decomposition, Independent Component Analysis, or any other representation of the covariance matrix using inferences about activity at missing electrode locations based on knowledge of the factor loadings of Principal Component Analysis.

In addition, images of brain regions activated by particular cognitive tasks or regions influenced by centrally active substances may be formed using a self norm to extract mean and standard deviation measures in a resting reference state and in the intra-task state. The effects of the task on the brain would then be shown by computing LORETA using the Z-scores of the task data Z-transformed relative to the self-norm.

Similarly, distributed inverse solution brain images may aid to visualize informational transactions in the frequency domain, which may be computed using transfer entropy to estimate the transmission of information and interaction among neuroanatomical regions at any frequency in the very narrow band power spectrum (e.g., from 0.39 to 50 Hz in 0.39 Hz intervals domain

Similarly, distributed inverse solution brain images of activity changes in the time domain may be computed for the transmission of information through and interaction among neuroanatomical regions at intervals in the time domain (e.g., in the millisecond range) across an analysis epoch of any event related potential relative to the pre-stimulus self-norm.

Finally, an MRI brain atlas may then be generated and stored in the QSL enabling anatomical identification of the brain image by interrogation of any voxel (e.g., with a cursor superimposed on the image) to allow a user to inspect and analyze the anatomical representations of brain activity voxel by voxel.

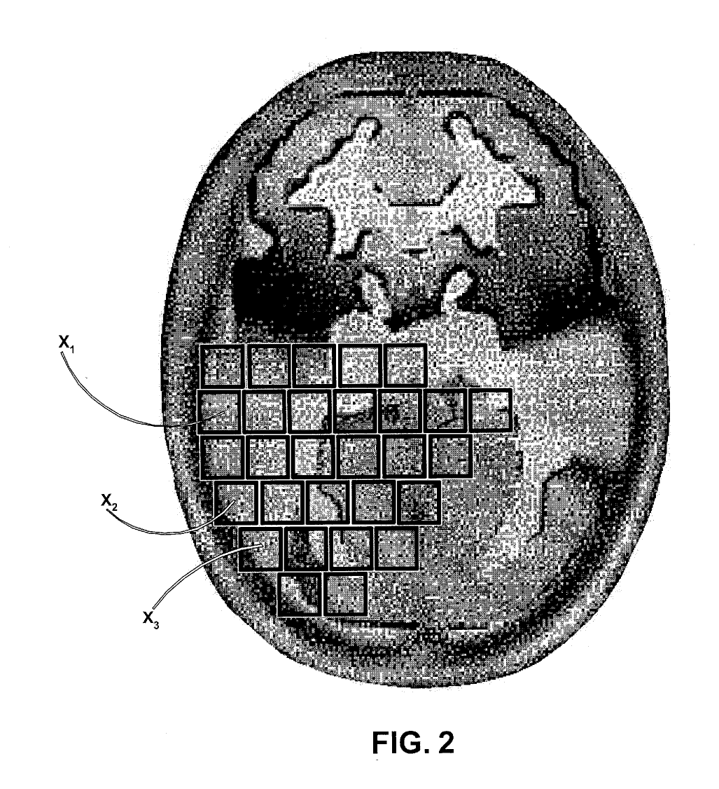

FIG. 2 shows an exemplary brain slice image on which is superimposed a plurality of 2D representations of voxels. For the purpose of illustrating the notion that the brain image is divided into a plurality of voxels, the voxels in FIG. 2 have been enlarged to an exaggerated degree. In actuality, the voxels used herein may be 5 mm cubes. Although for the sake of illustration, only a portion of the brain image has been divided into voxels, in actuality the present invention can divide the entire brain region into voxels, from the cerebral cortex down to the brain stem. Moreover, although illustrated in two-dimensional form in FIG. 2, it should be appreciated that the voxels are three-dimensional quantities that are calculated as such by the present invention. As shown in FIG. 2, each voxel represents a different ROI in the brain and is associated with its own Z score that signifies the extent to which that region of the brain exhibits brain activity that deviates from what is considered normal activity, as determined by a normative database. For example, voxels X1, X2, and X3 represent the brain activity occurring in three different ROIs in the brain. Moreover, as shown by the different degrees of shading in FIG. 2, the magnitude of the Z scores for each voxel can be shown visually by assigning different shades (or colors) to different voxels. Higher Z scores, for instance, can be associated with darker shading or brighter colors. Finally, even though the brain slice shown in FIG. 2 has been taken from a transverse perspective, the present invention is compatible with image slices taken from a coronal or sagittal perspective as well.

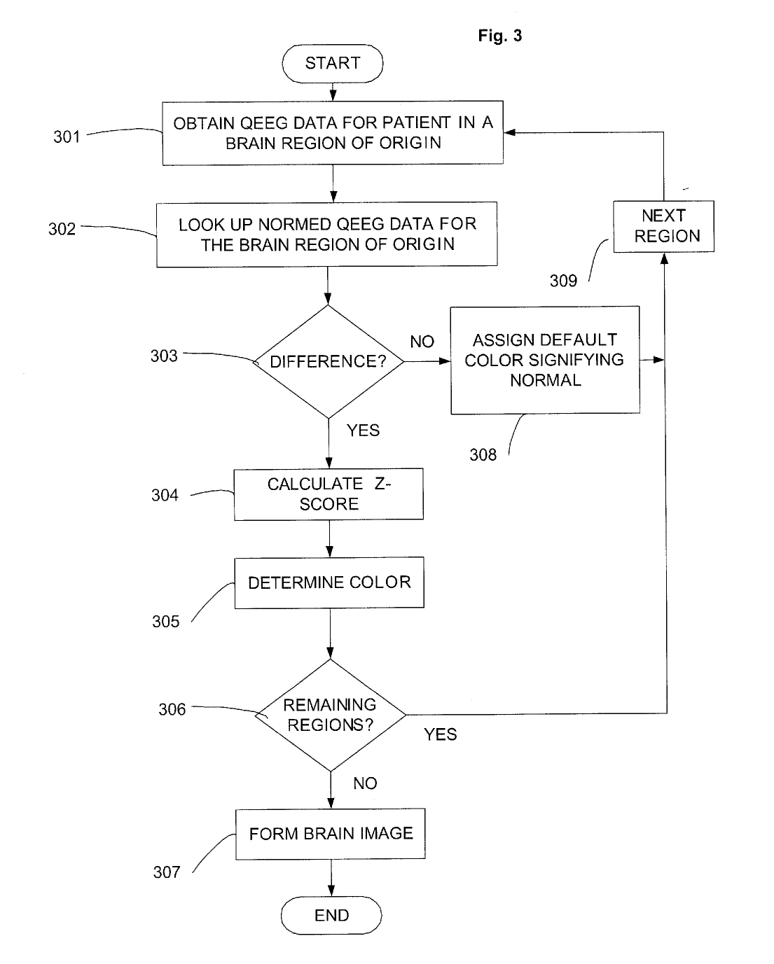

FIG. 3 illustrates a flow diagram of an exemplary method for generating an image of the brain containing visual indicia for highlighting which regions exhibit abnormal brain activity. The method may begin by having the processing unit 1 determine QEEG data for a particular brain region of origin (step 301). This determination may be done in real time, while the patient is connected via electrodes 2, or it may involve accessing pre-recorded QEEG data from any suitable recording medium. The QEEG data may be obtained in the manner discussed above, and the brain region to which it corresponds may be visually represented as a voxel or ROI comprised of a set of voxels. Next the processing unit 1 looks up from database 14 normed QEEG data for the voxel, region, or ROI in question. As explained above, the normed QEEG data may be a self-norm representing QEEG data taken from the patient 20 when the patient was not exhibiting any symptoms indicative of abnormal brain activity, or at some time when the patient was symptomatic, or it may be a population norm representing what the QEEG data for the region in question ought to be for a person of the age and gender of patient 20. If no difference exists between the QEEG data taken from patient 20 and that obtained from database 14, the method assigns to the voxel intended to represent the current region a color indicative of normal brain activity (e.g., green) (step 308) and proceeds to the next region of the brain (step 309).

If a difference does exist, then processing unit 1 will calculate a Z-score for the current region under consideration (step 304), and then it will assign to the associated voxel that visually represents the region a color indicative of the degree of brain activity abnormality as evidenced by the Z-score (e.g., red for increased and severely abnormal functioning, yellow for moderately abnormal functioning) or blue for moderately and turquoise for severely abnormal decreased function (step 305).

Processing unit 1 then determines whether any remaining brain regions require analysis (step 306). According to the exemplary embodiment, the entire volume of the brain, encompassing both cortical and subcortical regions and extending down to the brain stem, is divided into approximately 6,980 voxels, with each voxel being a 4 mm cube. In the exemplary embodiment of the present invention, every voxel is analyzed and color-coded to provide a visual marker of the degree of abnormality for that voxel.

The present invention is also consistent with a method in which only a subregion of the brain, for example, the frontal lobe, is analyzed and visually represented in this manner, or a method which truncates the brain to show only those regions whose standard score exceeds some positive (excess) or negative (deficit) threshold.

After all the brain regions of interest have been analyzed and their respective voxels color-coded, processing unit 1 forms a LORETA brain image composed collectively of these color-coded voxels. The processing unit 1 then outputs the color-coded brain image via I/O arrangement 12 (e.g., a display). Even though the present invention can analyze an entire brain volume in the manner described above and display a 3D image of the brain with its various regions colored in the manner just described, it need not display the entire brain, but can instead be instructed, through any suitable brain imaging techniques, to display only those voxels that are of interest. For instance, the displayed image may include just a slice image showing the brain activity in a specific plane.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for analyzing numerical values representative of at least a first portion of electrophysical signals produced by regions of a brain, the method comprising:
a) comparing a first selected one of the numerical values to control data including one of a self-norm and a population-norm associated with electrophysical activity in a brain region corresponding to a brain region of origin of the electrophysical activity on which the numerical value is based;
b) calculating a standard score for the brain region of origin based on the comparing; and
c) repeating steps a) and b) for a second selected one of the numerical values, wherein the regions of the brain include subcortical regions extending to a brain stem of the brain.

2. The method of claim 1, further comprising:
assigning one of an alphanumeric value for the standard score and a visual characteristic to the brain region of origin that is proportional to the standard score; and
superimposing the one of the alphanumeric value and the visual characteristic onto a predetermined image of the brain to form a statistically interpretable image of the brain.

3. The method of claim 1, wherein the visual characteristic of the brain region of origin visually represents an extent to which the standard score deviates from the control data.

4. The method of claim 3, wherein the visual characteristic includes a color for the brain region of origin, the color corresponding to a hue that is proportional to a probability of abnormality in the brain region of origin.

5. The method of claim 4, wherein the visual characteristic for the brain region of origin includes at least one voxel, the at least one voxel corresponding to a subcortical region of the brain.

6. The method of claim 1, wherein the control data includes a predetermined mean value and a standard deviation for the electrophysical activity in the brain region of origin, wherein the predetermined mean value is obtained from a database containing measures of a mean value and a standard deviation of QEEG variables for each of a plurality of regions of the brain.

7. The method of claim 6, wherein the QEEG variables include one of univariate and multivariate variables.

8. The method of claim 6, wherein the QEEG variables are derived from a population of persons spanning an age range from 6 to 90 years old with normal brain functions that do not present a symptom of a disease.

9. The method of claim 1, wherein the predetermined image of the brain includes one of a proportional brain space and a centimetric brain space.

10. The method of claim 1, further comprising, prior to step a), collecting the numerical values for the at least first portion of the brain.

11. The method of claim 10, wherein the collecting includes collecting the numerical values from a plurality of EEG scalp electrodes placed on a skull of a person.

12. A system for analyzing numerical values representative of at least a first portion of electrophysical signals produced by regions of a brain, the method comprising:
a) an arrangement for comparing a first selected one of the numerical values to control data including one of a self-norm and a population-norm associated with electrophysical activity in a brain region corresponding to a brain region of origin of the electrophysical activity on which the numerical value is based;
b) an arrangement for calculating a standard score for the brain region of origin based on the comparing; and
c) an arrangement for repeating steps a) and b) for a second selected one of the numerical values, wherein the regions of the brain include subcortical regions extending to a brain stem of the brain.

13. The system of claim 12, further comprising:
an arrangement for assigning one of an alphanumeric value for the standard score and a visual characteristic to the brain region of origin that is proportional to the standard score; and
an arrangement for superimposing the one of the alphanumeric value and the visual characteristic onto a predetermined image of the brain to form a statistically interpretable image of the brain.

14. The system of claim 13, wherein the visual characteristic of the brain region of origin visually represents an extent to which the standard score deviates from the control data.

15. The system of claim 14, wherein the visual characteristic includes a color for the brain region of origin, the color corresponding to a hue that is proportional to a probability of abnormality in the brain region of origin.

16. The system of claim 15, wherein the visual characteristic for the brain region of origin includes at least one voxel, the at least one voxel corresponds to a subcortical region of the brain.

17. The system of claim 12, wherein the control data includes a predetermined mean value and a standard deviation for the electrophysical activity in the brain region of origin, wherein the predetermined mean value is obtained from a database containing measures of a mean value and a standard deviation of QEEG variables for each of a plurality of regions of the brain.

18. The system of claim 17, wherein the QEEG variables include one of univariate and multivariate variables.

19. The system of claim 17, wherein the QEEG variables are derived from a population of persons spanning an age range from 6 to 90 years old with normal brain functions that do not present a symptom of a disease.

20. The system of claim 13, wherein the predetermined image of the brain includes one of a proportional brain space and a centimetric brain space.

21. A method for analyzing numerical values representative of at least a first portion of electrophysical signals produced by regions of a brain, the method comprising:
computing transfer entropy data corresponding to bi-directional informational electrical transactions at each of a plurality of frequency ranges between each of a plurality of regions of interest (ROI); and
transforming the transfer entropy data to Z-scores or standard scores relative to control normative data.

22. The method according to claim 21, wherein the transfer entropy data includes data corresponding to one of influences of each ROI transmitted to every other ROI, influences received at each ROI from all other ROIs, and the mutual information received in common by a first and a second ROIs from a third ROI.
